(19) United States
(12) Patent Application Publication
(10) Pub. No.: US 2008/0118417 A1
(43) Pub. Date: May 22, 2008

(54) Exothermic Personal Lubricant

(75) Inventor: Kevin Mallory, Albuquerque, NM (US)

Correspondence Address: PEACOCK MYERS, P.C., 201 THIRD STREET, N.W., SUITE 1340, ALBUQUERQUE, NM, 87102, US

(73) Assignee: Blaise Harrison, LLC, Albuquerque, NM (US)

(21) Appl. No.: 11/875,667

(22) Filed: Oct. 19, 2007

Related U.S. Application Data
(60) Provisional application No. 60/862,115, filed on Oct. 19, 2006.

(51) Int. Cl. B01J 4/00; A61M 35/00

(52) U.S. Cl. 422/236; 604/290

(57) ABSTRACT

A heat stimulating personal lubricant. A package and method for generating the heat stimulating personal lubricant is also disclosed.

### CROSS-REFERENCE TO RELATED APPLICATIONS

This application claims priority to U.S. Provisional Patent Application Ser. No. 60/862,115, entitled “Exothermic Personal Lubricant”, to Mallory et al., filed on Oct. 19, 2006, and the specification thereof is incorporated herein by reference.

### BACKGROUND OF THE INVENTION

1. Field of the Invention (Technical Field)

The present invention is an exothermic lubricant which releases heat through an exothermic chemical reaction.

2. Description of Related Art

There are many types of warming lubricants known in the art. These warming lubricants typically generate heat through the application of one-phase systems seeking water to generate heat. These lubricants depend on the existence of water in or around living tissue to initiate an exothermic reaction to generate the desired heat. In other words, the known art uses a technique that may render living tissue as a reaction vessel while generating heat. Thus, a minimum degree of moisture must exist in or around living tissue to initiate the warming sensation of known warming lubricants.

Other warming lubricants fail to control the range of heat produced since their warming sensation depends on the molar amount of water existing in or around the tissue at the time of application. Further, the known warming lubricants have some risk of human error in the applicative technique whereas the present invention prevents risk of human error in its applicative technique.

While known lubricants heat by using inner tissue moisture or surrounding water, the present invention releases water in a warm environment and utilizes two separate phases that react instantly together to produce the lubricant desired as well as the heat needed. The present invention generates the desired heat using reagents in a manner independent of the existence of water in the surrounding environment, and administers it safely to living tissue. In other words, the present invention does not depend on any amount of moisture to exist in the living tissue to initiate any of the reactions desired. Therefore, the present invention is very helpful for cases of severe dryness because it restores the vitality of the dry tissue.

In contrast to known warming lubricants, the present invention demonstrates full control over the amount of heat produced. Therefore, it is very suitable for heat sensitive reactants, as well as other reactants with optimum temperature to operate.

### BRIEF SUMMARY OF THE INVENTION

One embodiment of the present invention is a package for generating a heat stimulating lubricant comprising a first chamber holding a first reactant, a second chamber holding a second reactant, and at least one seal wherein the seal is located between the first chamber and the second chamber to keep the reactants from reacting with each other until the seal is broken. Upon the seal breaking and upon the reactants mixing, the reactants react, create an exothermic chemical reaction and generate a heat stimulating lubricant. The package preferably comprises a mixing chamber wherein the first reactant and second reactant mix and react to cause an exothermic reaction and a breakable seal allowing the lubricant to escape from the package. The first reactant of the package is preferably an acid and the second reactant is preferably a base. The first reactant also preferably comprises water. The package chambers are preferably made of a plastic or plastic-coated metal foil. At least one of the chambers of the package preferably comprises one or more of the following: a flavoring, a viscosity builder, a heat insulator, a tissue rejuvenator, a soothing agent, a dye and/or a botanical extract.

A second embodiment of the present invention is a method for using a heat stimulating personal lubricant comprising the steps of providing a package comprising two chambers and at least one seal, the first chamber comprising a first reactant and the second chamber comprising a second reactant, applying pressure to at least one of the chambers, breaking the seal, and allowing the first reactant to mix with the second reactant, causing an exothermic chemical reaction between the reactants and generating the heat stimulating lubricant, releasing the lubricant from the packaging, and applying the lubricant. The mixing step is preferably performed in a mixing chamber.

Another embodiment of the present invention is a heat-stimulating personal lubricant that has at least one acid reactant and at least one base reactant. These reactants react and create an exothermic chemical reaction and generate the heat stimulating lubricant. Preferably, at least one reactant comprises water and/or at least one polyol. The lubricant also preferably comprises one or more of the following: an insulating agent, a tissue rejuvenator, a soothing agent, a botanical extract, a dye, and or a flavoring.

An advantage of the present invention is that it soothes and lubricates, thereby stimulating, facilitating and relieving the pain of sexual intercourse (and/or similar act), turning it into a relaxing, pleasant experience with minimal irritation and/or discomfort. The lubricant may also be used on the skin as a warming lubricant.

Objects, advantages and novel features, and further scope of applicability of the present invention will be set forth in part in the detailed description to follow, taken in conjunction with the accompanying drawings, and in part will become apparent to those skilled in the art upon examination of the following, or may be learned by practice of the invention. The objects and advantages of the invention may be realized and attained by means of the instrumentalities and combinations particularly pointed out in the appended claims.

### BRIEF DESCRIPTION OF THE SEVERAL VIEWS OF THE DRAWINGS

The accompanying drawings in the attachment, which are incorporated into and form a part of the specification, illustrate one or more embodiments of the present invention and, together with the description, serve to explain the principles of the invention. The drawings are only for the purpose of illustrating one or more preferred embodiments of the invention and are not to be construed as limiting the invention. In the drawings:

FIG. 1 is a perspective view of a mixing and delivery package for the lubricant of the present invention;

FIG. 2 is a perspective view of an alternative embodiment of a mixing and delivery package for the lubricant of the present invention;

FIG. 3 is a perspective view of an alternative embodiment of a mixing and delivery package for the lubricant of the present invention;

FIG. 4 is a perspective view of an alternative embodiment of a mixing and delivery package for the lubricant of the present invention;

FIG. 5 is a perspective view of an alternative embodiment of a mixing and delivery package for the lubricant of the present invention;

FIG. 6 is a perspective view of an alternative embodiment of a mixing and delivery package for the lubricant of the present invention;

FIG. 7 is a perspective view of an alternative embodiment of a mixing and delivery package for the lubricant of the present invention; and

FIG. 8 is a perspective view of an alternative embodiment of a mixing and delivery package for the lubricant of the present invention.

### DETAILED DESCRIPTION OF THE INVENTION

The present invention is preferably a non-toxic liquid lubricant composition with an exothermic chemical reaction generating the composition of the lubricant (preferably an aqueous medium). The lubricant has a wide range of viscosities formed in a medium comprising reactants and other auxiliary reagents. The lubricant soothes and lubricates, thereby stimulating, facilitating and relieving the pain of sexual intercourse (and/or similar act), turning it into a relaxing, pleasant experience with minimal irritation and/or discomfort. The lubricant may also be used on the skin as a warming lubricant and is not limited to the uses described herein.

The terms “reactant” or “reactants”, as used throughout this specification, includes one or more chemicals which can be combined with one or more other chemicals to produce an exothermic reaction.

The reaction in this invention does not involve or require water from the human tissue as in other lubricants. Instead, the invention releases a controlled amount of water during the reaction. Thus, it is rendered safer then other warming lubricants since neither the epidermis nor the mucosa acts as a partner or a vessel for the reaction. The liquid product of the reaction is warm, preferably approximately 5 to 30 degrees Fahrenheit above ambient temperature, and retains and gently radiates such temperature for about 5 to 25 minutes from the time of application. It is also formulated to the pH environment of healthy living tissue, such as the skin, the vaginal cavity or other mucosa, and is a mild, non-irritant on healthy normal skin and/or mucosa.

The structure of the media in which the warming reaction occurs is selected to accommodate and safely deliver a wide variety of natural and/or synthetic extracts and drugs known to help soothe some epidermal and vaginal symptoms, including but not limited to, dysmenorrhea. It is also found to be suitable to deliver some antibiotics, moisturizing and lubricating agents.

The preferred reactants are compatible with spermicides including, but not limited to, certain Nonoxynol compounds and others such as methoxypolyoxyethyleneglycol 550 laurate.

The composition of the lubricant may also include anesthetics, including but not limited to benzocaine, lidocaine, benzyle alcohol, and the like. The inclusion of plant extracts, including but not limited to aloe vera extract, witch hazel, and the like, is also useful in achieving a soothing effect. Also suitable are certain soothing vegetable oils, including but not limited to olive, grape seed and jojoba oils.

The lubricant increases sexual stimulation and enhances sexual pleasure when used. However, it can also be used for relieving frostbite or chill. Females with sexual dysfunction and/or vaginal atrophy may find this warming lubricant useful in stimulating the tissue to easily accept hormone replacement, anti-inflammatory agents and/or analgesics.

The temperature released by the exothermic reaction is preferably controlled to be accurate within a range of approximately 1 to 2 degrees Celsius. This control feature allows the lubricant to be used as a heat vehicle when applying a wide variety of products that are heat sensitive, such as vitamins and certain compounds, to sensitive areas of the body.

The present invention is useful for human as well as animal use. The composition and method(s) of application are versatile. The invention may be used by itself, or in combination with other products or procedures, such as a self-heating kit useful in the delivery of treatment reagents and/or systems under controlled temperature(s) as needed in areas hard to reach, such as aural, bucal, rectum, nasal and vaginal cavities.

In contrast to other lubricants, the composition of the present invention is preferably substantially hydrous, containing water ranging from approximately 50% to 95% by weight.

### Detailed Chemical Description of the Exothermic Lubricant

The preferred composition of the present invention is described below.

1) Viscosity Builders/Lubricants. Besides water as a major component, the invention preferably comprises one or more types of polyols, preferably polyhydric alcohol and/or derivative, polyethylene glycol (PEG), propylene glycol and/or derivative and like compounds. The reaction matrix is mainly water plus one or more types of polyols to produce a system with reasonably high heat capacity, able to retain heat for a reasonable amount of time before the temperature drops down to ambient temperature.

2) Heat Insulators/Viscosity Builders. An insulating natural agent, including but not limited to, honey or similar function compounds, such as the mono-, di- and poly-sacharides may also be part of the lubricant's composition. Examples include but are not limited to glucose, fructose, sucrose, sorbitol, sorbose, amylum, pectin, xanthan gum and/or derivatives and/or the trichlorogalactosucrose (TGS) known as sucralose, and the like. Sole compound and/or articulate mixtures of the above may be used. A nonionic, water soluble ether of cellulose and ethylene oxide is also preferably part of the matrix mixture, such as the hydroxyethylcellulose series known as natrosols and/or derivatives and the like compounds.

3) Tissue Rejuvenator. A poly peptide substance such as collagen and/or water soluble proteins of high average molecular weight such as gelatin or the like can also be added to help repair damaged mucosa or tissue.

4) Soothing Agents. A soothing agent and/or a botanical extract is preferably added to enhance the ability of the lubricant to function and achieve the desired goals. Useful extracts include, but are not limited to, aloe vera and derivatives and/or natural fruit essential oils and/or extracts, including but not limited to cucumber, vanilla, strawberry, lemon, lime, cherry, black raspberry, orange, citrus, mango, pineapple, blueberry, licorice, cinnamon, peppermint and rose oil. A sole ingredient and/or a mixture may be used as needed.

5) Dyes. The lubricant may optionally include a suitable dye (preferably a FD&C dye) such as blue, green, red, orange, yellow and/or brown. The dye is added to the lubricant to identify the course of the reaction completion and/or to give the product a recognized appearance as desired.

6) Generation of Heat and Warming Effect. The heat and warming effects of the lubricant come from an acid-base (redox) exothermic reaction. The selection of an acid and/or a base depends on suitability and ability to instantly produce the desired heat as well as the nature of final reaction products and the safety impacts on the living tissue. Several preferred reactants are described below.

The lubricant comprises two parts, described as Part A and Part B. Part A includes, but is not limited to, the reaction matrix and optional additives as described above, plus the designated components (including but not limited to acid or combinations of acids) as selected for the heating reaction. Such acid(s) preferably include but are not limited to carboxylic, of the type mono-, di- or poly-carboxylic functional group(s) such as acetic, propionic, salicylic, valeric, vanillic, lactic, sorbic, glyceric, hippuric, benzoic, gluconic, malic, tartaric, glutaric, citric, and the like, but may also be of the amino carboxylic type, such as amino acetic acid (glycine), 2-amino glutaramic acid (glutamine), glutamic acid, folic acid, and the like. The monosodium salt of L-glutamic acid may also be used in the heat generating reaction due to its active carboxylic group. A suitable non-carboxylic acid, including but not limited to kojic acid or the like may be applied to Part A to enter the reaction as an acidulant or as a part of a buffer system. This part may or may not include the insulating reagent(s), botanical extracts and/or the dye(s).

Part B includes a reactant (preferably a base) that is completely or partially soluble in water, alcohol, ether and/or a mixture with or without the addition of one of the above mentioned polyols used herein as a thickener, solubilizer and/or aid for the heat transferring rate. The base reactant may include, but is not limited to, a hydroxide of one of the alkaline earth metal members of Ia and IIa of the periodic table, and/or the like reactants, preferably and including but not limited to potassium, sodium, magnesium, iron or calcium. Ammonium hydroxide (aqua ammonia) and/or ammonium bicarbonate and/or sodium carbonate or bicarbonate are preferable as they have been found to deliver the desired temperature using the proper molar ratio against the selected acid. The oxide salts of magnesium, calcium and sodium also were found to generate appreciable heat. Part B, in its composition, may or may not include certain amounts of viscosity builder, heat insulator, tissue rejuvenator, soothing agent and/or the dye as suitable. However, and in all cases, while selecting the proper reagents located in two separate reacting bodies, Parts A and B, they preferably exhibit one or more of the following attributes:

stability under normal conditions of temperature and pressure;

easy instant mix and easy application;

non-toxic, non-irritating reaction products to skin and/or mucosa;

heat stimulating and soothing effect;

easy to rinse or douche;

lubricating and moisturizing effect;

pH compatible with acceptable taste and/or aroma;

safe and feasible to deliver anti-inflammatory, antibiotic, spermicides, medicated oils, and botanical extracts;

environmentally friendly; and

economical with reasonable shelf life.

Formula Composition of the Exothermic Lubricant

The preferred composition of the lubricant of the present invention is described below:

Part A. The water percentage varies between approximately 20% to 90% by weight; and preferably between approximately 65% and 75% by weight. Nonionic water soluble ether of cellulose and ethylene oxide (cellulose derived polymer) comprises a percentage between approximately 0.1% and 10% by weight and acts as a viscosity builder and thickening agent, by providing pseudoplasticity and regulating the rheology and water retention agent to provide high efficiency and solubility in a saline system, suspending and protecting colloidal action, binding and stabilizing. Part A also comprises mono-, di- and/or poly-saccharide in a percentage ranging from approximately 1% to 20% by weight, including, but not limited to, glucose or a glucose derivative, fructose, sucrose or a sucrose derivative, amylum, pectin, xanthan gum or derivative in a percentage ranging from approximately 0.1% to 15% by weight. Polyethylene glycol (PEG) series, such as PEG 300, is a Part A component in a percentage ranging from approximately 3% to 15% by weight. The polyol percentage, such as propylene glycol, is between approximately 1% to 35% by weight. The tissue rejuvenator, including but not limited to a polypeptide substance such as collagen and/or gelatin may be in the range of approximately 0.1 to 25% in some applications. One or more of the reactive acid's type and category is partially or completely dissolved in Part A in a percentage by weight ranging from approximately 2% to 60%, the list including, but not limited to:

Monocarboxylic type:
Acetic acid
Propionic acid
Salicylic acid
n-Valeric acid
Vanillic acid
Glycine (amino acetic acid)
Lactic acid
Sorbic acid
Glyceric acid
Glutamine (2-amino-glutaramic acid)
Hippuric acid
Benzoic acid
Gluconic acid

Di-carboxylic type:
Malic acid
Folinic acid
Adipic acid
Tartaric acid (the D, L, DL and meso types)
Glutamic acid (amino acid)
Folic acid (amino acid)
Glutaric acid

Poly-carboxylic group acids
Citric acid

Non-carboxylic acid
Kojic acid (and the likes)

Natural flavors or fragrances or a combination of both, when added, are preferably in the range of between approximately 0.01% and 5%. The dye or combination of dyes are preferably in the range of approximately 0.00001% to 0.1% by weight or as needed.

Part B. Part B comprises the base reactant which is one or a combination of the following ingredients supported by polyhydric alcohol and/or a suitable polyol as described above. The base reactant is partially or completely introduced as a solution or a paste. The base reactant is in the range of approximately 2% to 60% by weight and the amount added in this part is calculated to be in the proper ratio against the acid or combination of acids applied in Part A. Such ratio is drawn from the reaction equation (s) and calculated so that it allows the proper amount of positively charged protons or radicals to deliver the pH range desired and suitable for the application. A pH chart for certain example acids and a temperature table are illustrative. The base reactants include, but are not limited to, the following components: ammonium, sodium, potassium, magnesium, iron and/or calcium hydroxides. The carbonate and/or bicarbonate salts of sodium and/or ammonium and oxide salts of sodium, magnesium and calcium may also be added to Part B as sole salts or in combination with the hydroxide and/or the carbonates. Part B in its composition may or may not include certain amounts of viscosity builders, heat insulator, tissue rejuvenator, soothing agent and/or the dye as suitable.

### Formula pH Range for the Exothermic Lubricant

The formula pH range varies according to how and where the lubricant is applied. For the vaginal cavity, it is preferable to be between approximately 4 and 6 pH. The pH of a healthy vaginal cavity can go up to approximately 5.7; however, the lubricant preferably needs to be in the acidic range to decrease the potential of vaginal infection as the pH rises. Thus, the preferred pH range for the vaginal cavity is in the range of approximately 4 to 6. In other technical applications, the pH range may be in the acidic, neutral or the alkaline range, or it may be buffered to achieve the desired goals.

### Combination of Part A and Part B—Mixing and Delivery Package

Preferably, parts A and parts B of the lubricant of the present invention are mixed at the point of use. Prior to use, parts A and B remain separated and are stored in a mixing and delivery package that provides storage and separation of parts A and B. The mixing and delivery package allows a user to mix parts A and B prior to use, and comprises a delivery nozzle or applicator after parts A and B have been mixed.

The mixing and delivery package for the lubricant of the present invention is preferably constructed of but not limited to a clear plastic resin material, plastic coated soft aluminum foil, or a similar material that is easily sealed and/or broken. The material of the package is preferably inert to any of the parts A and B, or when mixed together, and is preferably heat resistant to be able to retain the heat of the reaction when parts A and B are mixed.

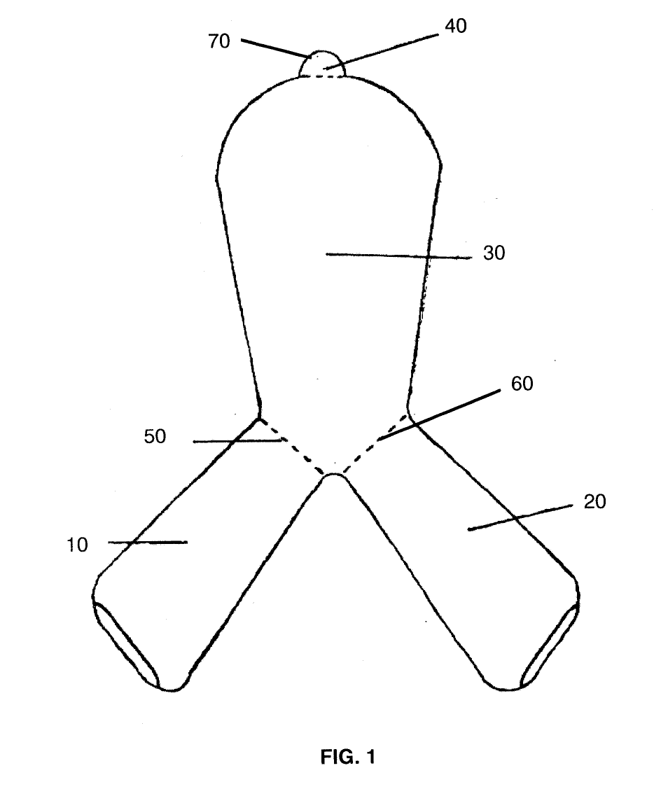

FIGS. 1-3 illustrate a three chamber design for the mixing and delivery package of the present invention. As shown in FIG. 1, elongated chambers 10 and 20 comprising parts A and B, respectively, are sealed from mixing chamber 30 and delivery nozzle 40 is sealed from mixing chamber 30. When a user squeezes chambers 10 and 20, seals 50 and 60 rupture, and parts A and B are combined and react to create the exothermic reaction and form the lubricant in mixing chamber 30. Seals 50 and 60 are ruptured when a user applies pressure to chambers 10 and 20. After parts A and B have been mixed to form the lubricant, a user removes delivery nozzle 40 (e.g. tearing it off) and ruptures seal 70 to allow the lubricant to be dispensed from the mixing and delivery package.

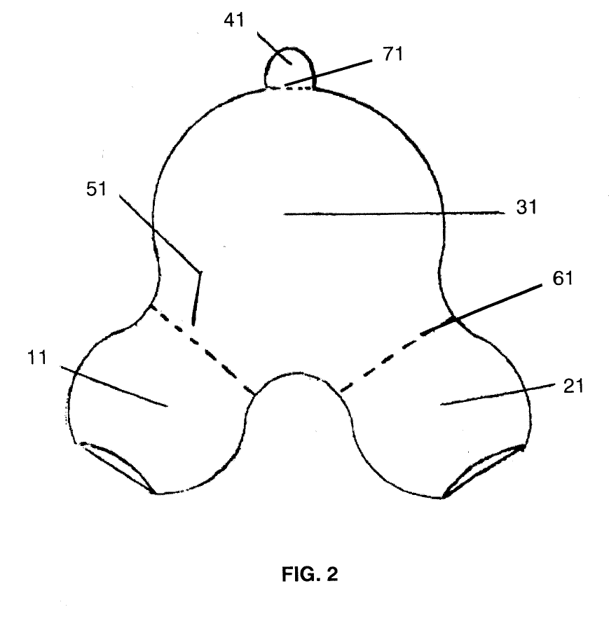

FIG. 2 illustrates rounded chambers 11 and 21 comprising parts A and B, respectively, which are sealed from mixing chamber 31 and delivery nozzle 41 is sealed from mixing chamber 31. When a user squeezes chambers 11 and 21, seals 51 and 61 rupture, and parts A and B are combined in mixing chamber 31 and react to create the exothermic reaction and form the lubricant. Seals 51 and 61 are ruptured when a user applies pressure to chambers 11 and 21. After parts A and B have been mixed to form the lubricant, the user removes delivery nozzle 41 (e.g. tearing it off) and ruptures seal 71 to allow the lubricant to be dispensed from the alternative mixing and delivery package shown in FIG. 2.

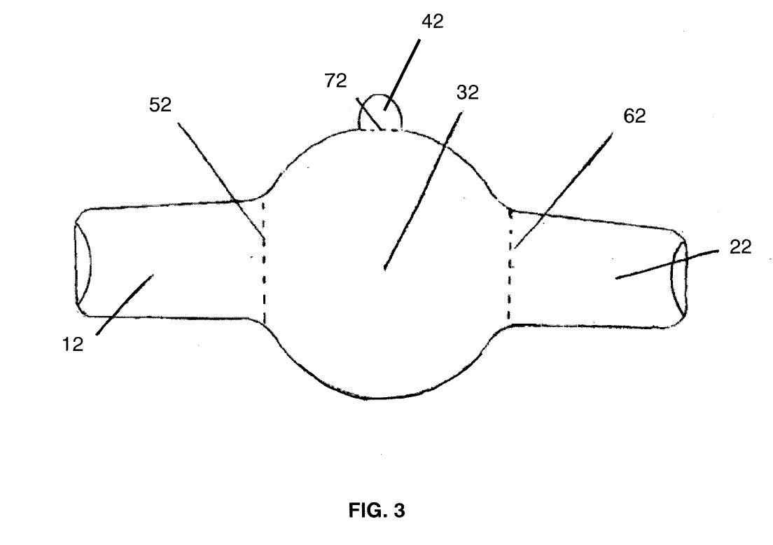

FIG. 3 illustrates rounded mixing chamber 32 with chambers 12 and 22 on opposite ends of mixing chamber 32. In this alternative embodiment, chambers 12 and 22 comprising parts A and B, respectively, which are sealed from mixing chamber 32 and delivery nozzle 42 is sealed from mixing chamber 32. When a user squeezes chambers 12 and 22, seals 52 and 62 rupture, and parts A and B are combined in mixing chamber 32 and react to create the exothermic reaction and form the lubricant. Seals 52 and 62 are ruptured when a user applies pressure to chambers 12 and 22. After parts A and B have been mixed to form the lubricant, the user removes delivery nozzle 42 (e.g. tearing it off) and ruptures seal 72 to allow the lubricant to be dispensed from the alternative mixing and delivery package shown in FIG. 3.

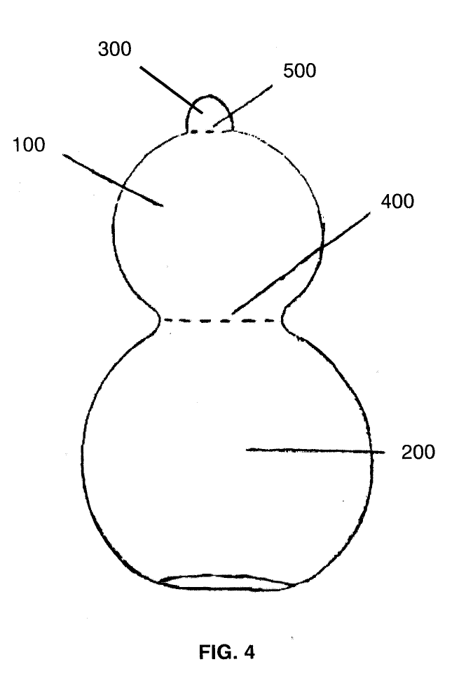

FIGS. 4-7 illustrate a two chamber design for the mixing and delivery package of the present invention. As shown in FIG. 4 chambers 100 and 200 comprising parts A and B, respectively, which are sealed from each other and delivery nozzle 300 is sealed from one of the chambers, either chamber 100 or chamber 200. When a user squeezes either chamber 100 or 200 or both, seal 400 ruptures, and parts A and B are combined and react to create the exothermic reaction and form the lubricant. Seal 400 ruptures when a user applies pressure to chamber 100 or 200 or both. After parts A and B have been mixed to form the lubricant, the user removes delivery nozzle 300 (e.g. tearing it off) and ruptures seal 500 to allow the lubricant to be dispensed from the alternative mixing and delivery package shown in FIG. 4. FIG. 4 also shows rounded chambers 100 and 200 that are stacked one on top of the other.

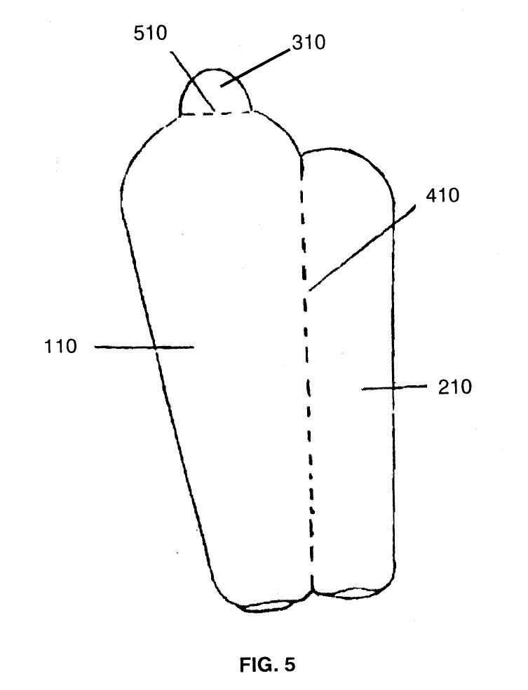

FIG. 5 illustrates another embodiment of the present invention where chambers 110 and 210 are elongated and located side-by-side with seal 410 as a separation. In this embodiment, chambers 110 and 210 comprising parts A and B, respectively, which are sealed from each other and delivery nozzle 310 is sealed from one of the chambers, either chamber 110 or chamber 210. When a user squeezes either chamber 110 or 210 or both, seal 410 ruptures, and parts A and B are combined and react to create the exothermic reaction and form the lubricant. Seal 410 ruptures when a user applies pressure to chamber 110 or 210 or both. After parts A and B have been mixed to form the lubricant, the user removes delivery nozzle 310 (e.g. tearing it off) and ruptures seal 510 to allow the lubricant to be dispensed from the alternative mixing and delivery package shown in FIG. 5.

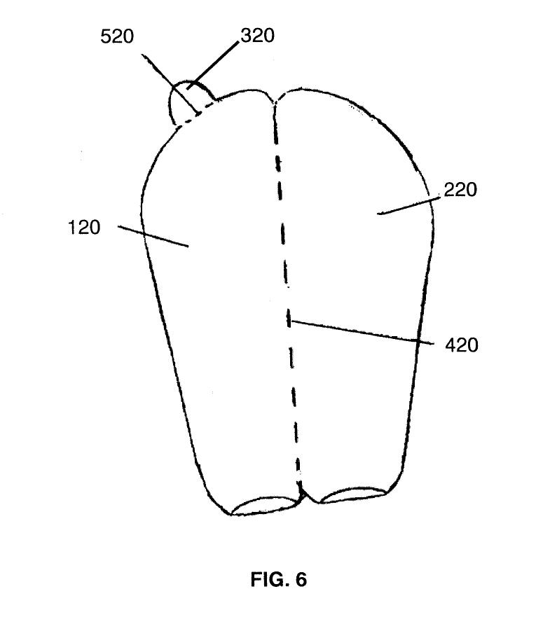

FIG. 6 illustrates yet another embodiment of the present invention where chambers 120 and 220 have a rounded and elongated shape and are located side-by-side with seal 420 as a separation. In this embodiment, chambers 120 and 220 comprising parts A and B, respectively, which are sealed from each other and delivery nozzle 320 is sealed from one of the chambers, either chamber 120 or chamber 220. When a user squeezes either chamber 120 or 220 or both, seal 420 ruptures, and parts A and B are combined and react to create the exothermic reaction and form the lubricant. Seal 420 ruptures when a user applies pressure to chamber 120 or 220 or both. After parts A and B have been mixed to form the lubricant, the user removes delivery nozzle 320 (e.g. tearing it off) and ruptures seal 520 to allow the lubricant to be dispensed from the alternative mixing and delivery package shown in FIG. 6.

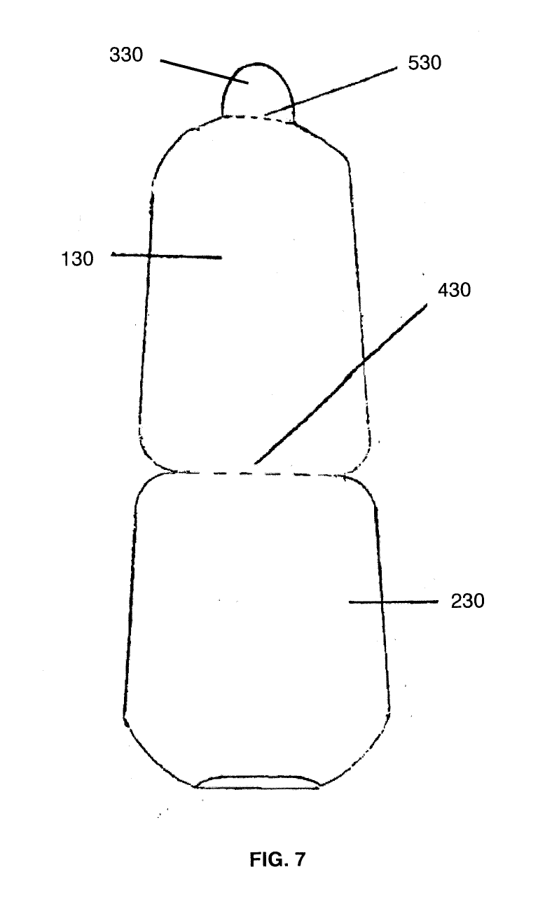

FIG. 7 illustrates an embodiment of the present invention where chambers 130 and 230 have a rectangular shape and are stacked one on top of the other with seal 430 as a separation. In this embodiment, chambers 130 and 230 comprising parts A and B, respectively, which are sealed from each other and delivery nozzle 330 is sealed from one of the chambers, either chamber 130 or chamber 230. When a user squeezes either chamber 130 or 230 or both, seal 430 ruptures, and parts A and B are combined and react to create the exothermic reaction and form the lubricant. Seal 430 ruptures when a user applies pressure to chamber 130 or 230 or both. After parts A and B have been mixed to form the lubricant, the user removes delivery nozzle 330 (e.g. tearing it off) and ruptures seal 530 to allow the lubricant to be dispensed from the alternative mixing and delivery package shown in FIG. 7.

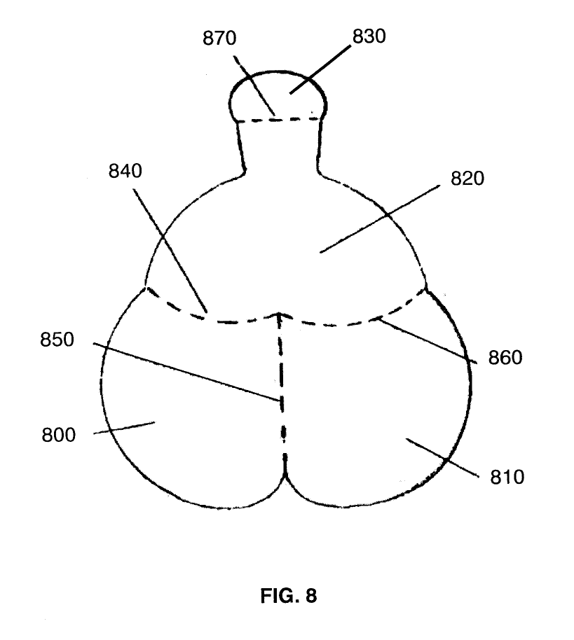

FIG. 8 illustrates a three-chamber design for the mixing and delivery package wherein chambers 800 and 810 comprising parts A and B, respectively, which are sealed from mixing chamber 820 and from each other. Delivery nozzle 830 is sealed from mixing chamber 820. When a user squeezes chambers 800 and 810, seals 840, 850 and 860 rupture, and parts A and B are combined in mixing chamber 820 and react to create the exothermic reaction and form the lubricant. Seals 840, 850 and 860 rupture when a user applies pressure to chambers 800 and 810. After parts A and B have been mixed to form the lubricant, the user removes delivery nozzle 830 (e.g. tearing it off) and ruptures seal 870 to allow the lubricant to be dispensed from the alternative mixing and delivery package shown in FIG. 8.

The shape of the mixing and delivery package may be, but is not limited to the following shapes: spherical, round, oval, oblong, rectangular, square, triangular, or any shape suitable for keeping parts A and B initially separated, then combined, and then dispensed.

In another embodiment, the seals of the mixing and delivery package are made of a material including but not limited to laminate, film, sheet or tubular material. Alternatively, commercially available seals may be utilized.

Although the invention has been described in detail with particular reference to these preferred embodiments, other embodiments can achieve the same results. Variations and modifications of the present invention will be obvious to those skilled in the art and it is intended to cover all such modifications and equivalents. The entire disclosures of all references, applications, patents, and publications cited above and/or in the attachments, and of the corresponding application(s), are hereby incorporated by reference. The reactants, flavoring, materials, components, etc., set forth in Ser. No. 60/862,115 are applicable to the various embodiments set forth herein.

1. A package for generating a heat stimulating lubricant comprising:
a first chamber holding a first reactant;
a second chamber holding a second reactant; and
at least one seal wherein said seal is located between said first chamber and said second chamber to keep said reactants from reacting with each other until said seal is broken; and
upon said seal breaking and upon said reactants mixing, said reactants reacting and creating an exothermic chemical reaction and generating said heat stimulating lubricant.

2. The package of claim 1 further comprising a mixing chamber wherein said first reactant and second reactant mix and reacts to cause an exothermic reaction.

3. The package of claim 1 further comprising a breakable seal allowing said lubricant to escape from said package.

4. (canceled)

5. The package of claim 1 wherein said at least one chamber comprise a plastic.

6. The package of claim 1 wherein said at least one chamber comprise plastic-coated metal foil.

7. The package of claim 1 wherein said first reactant comprises water.

8. The package of claim 1 wherein at least one of said chambers further comprises a flavoring.

9. The package of claim 1 wherein at least one of said chambers further comprises a viscosity builder.

10. The package of claim 1 wherein at least one of said chambers further comprises a heat insulator.

11-14. (canceled)

15. A method for using a heat stimulating personal lubricant comprising the steps of:
providing a package comprising two chambers and at least one seal, the first chamber comprising a first reactant and the second chamber comprising a second reactant;
applying pressure to at least one of the chambers, breaking the seal, and allowing the first reactant to mix with the second reactant;
causing an exothermic chemical reaction between the reactants and generating the heat stimulating lubricant;
releasing the lubricant from the packaging; and
applying the lubricant.

16. The method of claim 15 wherein the mixing is performed in a mixing chamber.

17-26. (canceled)
